(19) United States
(12) Patent Application Publication
(10) Pub. No.: US 2004/0171910 A1
(43) Pub. Date: Sep. 2, 2004

(54) Sexually stimulating panty insert

(76) Inventor: Robin B. Moore-Steele, Meridian, MS (US)

Correspondence Address: Robin B. Moore, P.O. Box 730, Marion, MS, 39342, US

(21) Appl. No.: 10/373,831

(22) Filed: Feb. 27, 2003

(51) Int. Cl. A61F 5/00

(52) U.S. Cl. 600/38

(57) ABSTRACT

A panty insert for sexually stimulating a female wearer. The panty insert includes a cover sheet and a backing sheet secured to the cover sheet. A number of stimulating members are positioned between the cover sheet and the backing sheet. One pair of the stimulating members is disposed so as to squeeze the labia minus of a user together and another one of the stimulating members is disposed so as to rub the clitoris the user.

### FIELD OF THE INVENTION

The present invention relates generally to medical apparatus and, more particularly, to pads for external application having a specific design, shape or structural feature.

### BACKGROUND OF THE INVENTION

Apparatus for massaging the genitals of a female for therapeutic or erotic purposes are well known. Many of these apparatus are electrically powered or require the constant attention of the hands of a user to obtain the desired effect. Of course, being removed from an electrical current source or being unable to use one's hands can cause difficulties in using some massaging apparatus. A need presently exists, therefore, for a manually operated device for massaging the genitals of a female user in a hands-free manner.

### SUMMARY OF THE INVENTION

In light of the problems associated with the prior art massaging apparatus, it is a principal object of the invention to device that can massage the genitals of a female user without bringing hands, electricity, or medications into play. The device can be discretely worn in the crotch portion of an undergarment like a woman's panties. The device covers the groin, extending over and around the genitals, perineum, and anus of a user. In use, portions of the device rub against the clitoris and labia during leg movement to cause sexual stimulation in a wearer and, perhaps, sexual satisfaction in her mate should circumstances permit.

It is another object of the invention to provide a stimulating device that is relatively small, thin, and unobtrusive and can be worn with loose or tight-fitting, outer garments of all types.

It is another object of the invention to provide a stimulating device of the type described that is easy and intuitive to use, requiring little, if any, instruction to employ. The device may be donned and doffed without removing any clothing. Further, a user need not modify any of her clothing to use the device, and use of the device will not harm her clothing.

It is a further object of the invention to provide a stimulating device of the type described that women of varying height, weight, and stature can use. Thus, the device can be universally employed and is of the one size fits all-type.

It is an object of the invention to provide improved elements and arrangements thereof in a sexually stimulating panty insert for the purposes described which is lightweight in construction, inexpensive to manufacture, and dependable in use.

Briefly, the sexually stimulating panty insert in accordance with this invention achieves the intended objects by featuring a cover sheet and a backing sheet secured to the cover sheet. A number of stimulating members are positioned between the cover sheet and the backing sheet. One pair of the stimulating members is disposed so as to squeeze the labia minus of a user together and another one of the stimulating members is disposed so as to rub the clitoris the user. A number of adhesive patches are provided on the backing sheet to secure the panty insert within an undergarment during use.

The foregoing and other objects, features and advantages of the present invention will become readily apparent upon further review of the following detailed description of the preferred embodiment as illustrated in the accompanying drawings.

### BRIEF DESCRIPTION OF THE DRAWINGS

The present invention may be more readily described with reference to the accompanying drawings, in which:

FIG. 1 is a perspective view of a sexually stimulating panty insert in accordance with the present invention having portions broken away to reveal details thereof.

FIG. 2 is a side elevational view of the panty insert of FIG. 1 with its release liners partially pulled therefrom.

FIG. 3 is a front view of the panty insert.

FIG. 4 is a diagrammatic view showing the positioning of the stimulating members when engaged with the body of a female user.

FIG. 5 is a cross-sectional view taken along line 5-5 of FIG. 3.

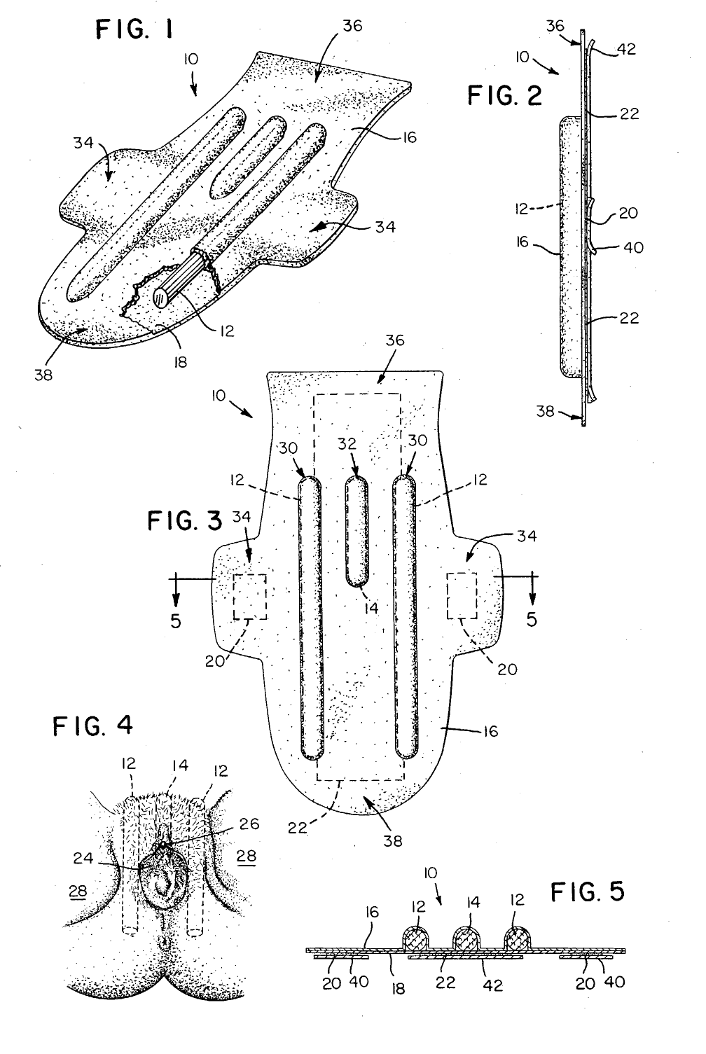

Similar reference characters denote corresponding features consistently throughout the accompanying drawings.

### DETAILED DESCRIPTION OF THE PREFERRED EMBODIMENT

Referring now to the FIGS., a sexually stimulating panty insert in accordance with the present invention is shown at 10. Panty insert 10 includes a number of stimulating members 12 and 14 positioned side by side between a permeable cover sheet 16 and an impermeable backing sheet 18. Backing sheet 18 is provided with a number of adhesive patches 20 and 22 for securing panty insert 10 within the crotch portion of an undergarment like a pair of panties (not shown). While disposed within the undergarment, stimulating members 12 and 14 respectively squeeze the labia minus 24 and rub against the clitoris 26 of the wearer during movement of her legs 28 to cause sexual stimulation in the wearer.

Each of the stimulating members 12 and 14 preferably comprises a short piece of cotton cording. Alternatively, members 12 and 14 may be formed of foam rubber or even a low density/high loft material like cotton wadding or wood pulp fiber. Low density/high loft materials, of course, offer the capability of quickly absorbing and distributing liquids. Members 12 and 14 have a diameter of about 0.4 inches (1.0 cm) and lengths of 6.0 inches (15.0 cm) and 2.4 inches (6.0 cm) respectively. Members 12 and 14 are spaced about 0.5 inches (1.25 cm) apart in a parallel arrangement with their front ends 30 and 32 in alignment with one another.

Backing sheet 18, preferably a thin film of polyethylene or polyvinyl, provides a moisture barrier for preventing liquids from passing through panty insert 10. Backing sheet 18 is adhesively bonded to the bottoms of stimulating members 12 and 14. As backing sheet 18 has somewhat larger dimensions than those of members 12 and 14 when arrayed side by side, backing sheet 18 extends outwardly from all sides of members 12 and 14 when secured thereto.

The dimensions of cover sheet 16 coincide with those of backing sheet 18 so that it too extends outwardly from all sides of stimulating members 12 and 14 when placed in registry with backing sheet 18 as shown in the FIGS. Preferably, the periphery of cover sheet 16, comprising a nonwoven, polymeric material such as rayon, is attached by a suitable adhesive or by heat- sealing to the adjacent portions of backing sheet 18. Thus, cover sheet 16 need not be bonded directly to stimulating members but may, rather, “float” over it.

Embossments (not shown) may be impressed in cover sheet 16. Between the embossments, cover sheet 16 is somewhat lofted in rounded, cushion-like contours where portions of stimulating members 12 and 14 press against cover sheet 16. The lofted areas may make panty insert 10 more comfortable, permitting such to be worn for extended periods of time.

Cover sheet 16 and backing sheet 18 are dimensioned to provide panty insert 10 with side flaps or wings 34 capable of being partially folded around the narrowed crotch portion of a pair of panties or other undergarment. Wings 34 extend outwardly from the opposite sides of panty insert 10 adjacent the midpoints of stimulating members 12. Additionally, cover sheet 16 and backing sheet 18 are dimensioned to provide an forward extension 36 and a rearward extension 38 that project outwardly from panty insert 10 adjacent the opposite ends of stimulating members 12.

The outside of backing sheet 18 is provided with spaced-apart adhesive patches 20 and 22 that serve as means for attaching panty insert 10 to an undergarment. As shown, adhesive patches 20 are located on wings 34. Adhesive patch 22, however, is disposed on backing sheet 18 between patches 20 and extends from forward extension 36 to rearward extension 38. Preferably, adhesive patches 20 and 22 comprise a nontoxic, contact-type glue that, after a period of use, may be pulled from an undergarment while leaving no residue on the undergarment.

Removable release liners 40 and 42 cover adhesive patches 20 and 22 in order to keep the adhesive from becoming contaminated prior to use. Release liners 40 and 42 are formed of paper having at least one surface treated as with silicone for ready detachment from adhesive patches 20 and 22. Of course, release liners 40 and 42 are retained on adhesive patches 20 and 22 until an individual detaches them while panty insert 10 is made ready for use.

Panty insert 10 can be made with conventional equipment in a continuous production process. Preferably, the material that is to form cover sheet 16 is first fed from a roll. (This rolled material has a constant width that is somewhat wider than that of stimulating members 12 and 14 when arrayed side by side.) As the cover sheet material is advanced, stimulating members 12 and 14 are cut to the desired length and placed on the cover sheet material. Next, the material that is to form backing sheet 18 is fed from a roll and brought into engagement with stimulating members 12 and 14 and the material that is to form cover sheet 16. By means of an adhesive previously applied to the backing sheet material, the backing sheet material is affixed to stimulating members 12 and 14 and to the cover sheet material around the periphery of stimulating members 12 and 14. After this operation has been completed, the exposed backing sheet material has adhesive patches 20 and 22 applied thereto. Panty insert 10 is then cut to shape and is severed from the selvage of the joined backing and cover sheet stock materials.

If cover sheet 16 is made of plastic material such as nonwoven rayon, it may be heat sealed to backing sheet 18. In this method of construction, adhesive is not needed to secure cover sheet 16 and backing sheet 18 together. Adhesive, however, may still be employed on stimulating members 12 and 14 to secure backing sheet 18 thereto.

Release liners 40 and 42 are next secured to adhesive patches 20 and 22. As shown, release liners 40 and 42 extend slightly beyond the peripheries of adhesive patches 20 and 22 so that, at the time of use, release liners 40 and 42 may be easily grasped and pulled from the adhesive patches 20 and 22.

In an alternative method of construction, cover sheet 16 and backing sheet 18 are formed of cotton cloth and massaging inserts 12 and 14 are formed from cotton cording. After cutting the cloth and cording to size, the resulting parts are sewn together to produce a panty insert 10 of great durability. Such an insert 10 can be washed with a conventional detergent after use, dried, and reused.

Regardless of the method of construction employed, panty insert 10 has an overall thickness of about 0.5 inches (1.25 cm) and is capable of bending to conform to the body of a wearer and provide a stimulating effect as the wearer moves about or even sits. Due to the unique configuration of stimulating members 12 and 14, panty insert 10 is easily attached to the narrowed crotch portion of an undergarment such as a pair of panties and is unobtrusive even when a tight-fitting outer garment is worn. After use, panty insert 10 may be easily removed from the narrowed crotch portion of the undergarment and conveniently discarded.

While the invention has been described with a high degree of particularity, it will be appreciated by those skilled in the art that modifications may be made thereto. For example, buttons, clasps, and hook and loop fasteners may be substituted for adhesive patches 20 and 22, though none of these is thought to be as convenient to use. Further, if desired, the ends of stimulating members 12 may be brought into contact with one another to form a ring that can fully encircle the genitals of a wearer to increase the desired massaging effect. Therefore, it is to be understood that the present invention is not limited to the sole embodiment described above, but encompasses any and all embodiments within the scope of the following claims.

I claim:

1. A sexually stimulating panty insert, comprising:
a cover sheet;
a backing sheet being secured to said cover sheet; and,
a plurality of stimulating members positioned between said cover sheet and said backing sheet, a pair of said stimulating members being disposed so as to squeeze the labia minus of a user together and another of said stimulating members being disposed so as to rub the clitoris the user.

2. The panty insert according to claim 1 further comprising at least one adhesive patch on said backing sheet for securing said panty insert within an undergarment.

3. A sexually stimulating panty insert, comprising:
a liquid pervious cover sheet;
a liquid impervious backing sheet being secured to said cover sheet;
an adhesive patch on said backing sheet for securing said panty insert within an undergarment; and,
a plurality of stimulating members positioned between said cover sheet and said backing sheet, said stimulating members being elongated cylinders positioned in a spaced apart, parallel relationship to one another, a pair of said stimulating members being disposed so as to squeeze the labia minus of a user together and another of said stimulating members being disposed so as to rub the clitoris the user.

4. The panty insert of claim 3 wherein at least one of said stimulating members comprises a length of cording.

5. A sexually stimulating panty insert, comprising:
a liquid pervious cover sheet;
a liquid impervious backing sheet in registry with said cover sheet and being secured thereto;
a plurality of stimulating members being positioned parallel to one another between said cover sheet and said backing sheet, one pair of said stimulating members being disposed on opposite sides of said backing sheet and each having a first length, another of said stimulating members being disposed between said pair of said stimulating members and having a second length being less than said first length;
said cover sheet and said backing sheet being provided with a pair of wings that extend outwardly therefrom on opposite sides of said stimulating members; and,
a pair of adhesive patches each being respectively disposed on one of said wings for securing said panty insert within an undergarment.
